(19) United States
(12) Patent Application Publication
(10) Pub. No.: US 2025/0345989 A1
(43) Pub. Date: Nov. 13, 2025

(54) 3-D PRINTED MULTI-ORGAN-ON-A-CHIP

(71) Applicant: Phase, Inc., Cornelius, NC (US)

(72) Inventor: Jeffrey Patrick SCHULTZ, Huntersville, NC (US)

(73) Assignee: Phase, Inc., Cornelius, NC (US)

(21) Appl. No.: 18/854,339

(22) PCT Filed: Apr. 5, 2023
(86) PCT No.: PCT/US2023/065371

§ 371 Date: Oct. 4, 2024

Related U.S. Application Data
(60) Provisional application No. 63/327,426, filed on Apr. 5, 2022.

(51) Int. Cl. B29C 64/124; B29K 83/00; B29K 105/00; B29L 31/00; B33Y 10/00; B33Y 80/00; C12N 5/00

(52) U.S. Cl. CPC B29C 64/124; B33Y 10/00; B33Y 80/00; C12N 5/0062; B29K 2083/00; B29K 2105/0014; B29L 2031/753; C12N 2533/40

(57) ABSTRACT

A process to additively manufacture a three dimensional part includes the steps of sequentially forming layers of a thermally curable mixture of polydimethylsiloxane (PDMS) and a curing agent, selectively thermally curing the individual layer by heating the layer with a focused electromagnetic radiation where less than 60% of the electromagnetic radiation passes through 1 micron of the heat curable mixture. The three dimensional part is then cleaned of any uncured heat curable mixture.

### CROSS REFERENCES AND PRIORITIES

This application claims priority from U.S. Provisional Patent Application No. 63/327,426 filed on 5 Apr. 2022, the teachings of which are incorporated by reference in their entirety.

### UNITED STATES GOVERNMENT RIGHTS

THIS INVENTION WAS MADE WITH GOVERNMENT SUPPORT UNDER 1R43TR003968-01 AWARDED BY THE NATIONAL INSTITUTES OF HEALTH. THE GOVERNMENT HAS CERTAIN RIGHTS IN THE INVENTION.

### BACKGROUND

Additive Manufacturing (AM), also known as three-dimensional (3D) printing has received much attention for printing optics and microfluidic structures.

Much of this work has focused on polydimethylsiloxane (PDMS). It is known as having unusual rheological properties, optically clear, non-toxic and non-flammable.

A review of such processes and their limitations are provided in United States Patent Application No. 2020/0030879 to Liang, et al., titled LASER-ASSISTED ADDITIVE MANUFACTURING OF OPTICS USING HEAT CURABLE MATERIALS, the teaching of which are incorporated by reference herein in their entirety.

Ultraviolet (UV) curing is considered the “go to” approach for manufacturing parts from PDMS. There is a large body of work attempting to change the chemistry or accelerate the hardening of PDMS. This is done by adding photo-initiators. However, these parts are considered inferior by the research community which wants the traditional PDMS thermally cured with a traditional curing agent to conduct its research. A need therefore exists to make PDMS parts with features less than 200 microns by thermal curing without the addition of UV absorbers or activators or photo-initiators.

Many processes use ultraviolet light to cure or polymerize the components of a mixture. According to Liang, the UV curing leaves the article yellowish.

UV curing is typically accomplished by photo-initiating a reaction caused by exposure to UV light in the UVB (280-320 nm) or UVA (320-395 nm) spectra. PDMS does not absorb light in these ranges and therefore a UV absorbing additive is required. This adds another component to the PDMS part.

One UV curable PDMS resin with properties similar to Sylgard™ 184 has been formulated, however feature resolution is limited to 200 micrometers or greater and the resin contains catalysts for the UV curing which have an unknown toxicity to various cells.

Extrusion based processes exist, but feature resolution and print times limit these applications.

Riahi, et al in their article titled “Fabrication of 3D microfluidic structure with direct selective laser baking of PDMS”, Rapid Prototyping Journal 25/4 (2019) p 775-780; used CO₂ laser having a wavelength of 10.6 micrometer (10,600 nm) to cure a mixture of PolyDiMethyl Siloxane and a hardener in an additive manufacturing layer-by-layer process.

Riahi's process sequentially lowered the part into a vat of PDMS mixture with the PDMS re-coated across the top of the part to form the next layer which was then thermally cured.

According to Riahi, the curing depth of PDMS with the laser was around 200 micrometers. This large curing depth is self-reportedly associated with the limited accuracy of the VAT POLYMERIZATION 3D printing techniques.

There exists therefore a need for a 3D printing process for heat curable resins of much thinner layers that does not rely upon a vat for making the layers.

### SUMMARY

A method of additively manufacturing a heat cured article from a thermally curable mixture comprising PDMS and a thermal curing agent. The method is disclosed as comprising the creation of a cured base layer of PDMS (L0) comprising the thermally curable mixture; forming a heat curable current layer (LN) comprising the thermally curable mixture having a heat curable current layer thickness (TN) on a preceding layer (L(N−1)) and then curing at least a portion of the heat curable current layer by selectively applying an incident electromagnetic radiation energy to heat at least a portion of the heat curable current layer and optionally leaving an uncured portion of the heat curable current layer, wherein less than 50% of the incident electromagnetic radiation energy is transmittable through 1 micron of the heat curable mixture.

It is further disclosed to repeat the forming and curing steps until the heat cured article is built; and then removing the uncured portions from inside and around the heat cured article.

It is further disclosed that the incident electromagnetic radiation energy may have wavelength in the range of 9.2 to 9.4 micrometers.

The specification also disclose that the heat curable mixture may comprise a PDMS weight percent and a thermal curing agent weight percent totaling 100 weight percent.

Also disclosed is that the heat cured article may be capable of transmitting at least 90% of the incident electromagnetic radiation having a wavelength in the range of 280 to 395 nanometers per micrometer of the heat cured article.

The specification also discloses an article of manufacture comprising cured PDMS, wherein the article has a channel having at least one dimension relative to the base layer selected from the group consisting of a height and a width that is less than 200 micrometers.

It is also disclosed that the at least one dimension could be less than 150 micrometers, less than 100 micrometers. less than 50 micrometers. or less than 25 micrometers.

The article is multi-layered and may comprise a layer of cured PDMS having a thickness in a range selected from the group consisting of 1 micrometer to 200 micrometers, 1 micrometer to 150 micrometers, 1 micrometer to 100 micrometers, 1 micrometer to 50 micrometers.

The article may also comprise a base layer (L0) of at least 50 micrometers.

It is further disclosed that the heat curable mixture of the heat cured PDMS article be void of a photo-initiator.

### BRIEF DESCRIPTION OF FIGURES

FIG. 1 is an apparatus to practice the invented process.

FIG. 2 is a CAD drawing of a multi-layer PDMS article.

FIG. 3 is a thermally cured multi-layer PDMS article made according to this invention.

FIG. 4 is a pictorial image of the layer forming step process on the left with an image of the apparatus on the right.

FIG. 5A is a pictorial image of the curing step of the process

FIG. 5B an image of the apparatus of FIG. 5A.

FIG. 6A is a pictorial image of the completed and spin rinsed product made by the process.

FIG. 6B is an image of the actual product on the apparatus on the right.

FIG. 7A and FIG. 7B depicts the optional addition of bioinks into cavities of the article and subsequent thermal curing of a PDMS lid layer on the top of the PDMS article.

FIG. 8 is an article manufactured by the process steps of this invention.

FIG. 9 is the transmittance of the electromagnetic radiation per micron of PDMS as a function of the wavelength of the electromagnetic radiation.

### DETAILED DESCRIPTION

The method disclosed herein is based upon the research done attempting to print a microfluidic part by sequentially thermally curing layers of PDMS (polydimethylsiloxane) and a thermal curing agent (hardener) without the addition of electromagnetic radiation adsorbers such as UV activators or photo-initiators.

One brand of this PDMS is known as Sylgard™M 184, available from DOW Chemical, Midland, Michigan, USA. According to the Material Safety Data Sheet, the base resin contains 0.5 wt % xylene, 0.2 wt % ethylbenzene, >60 wt % dimethylvinyl-terminated dimethyl siloxane, 30 to 60 wt % dimethylvinylated and trimethylated silica and 1 to 5 wt % tetra (trimethylsiloxy) silane.

The two component system is known as Dow Sylgard™ 184 Silicone Elastomer Base and Dow Sylgard™ 184 Silicone Elastomer Curing Agent. The curing agent Dimethyl, methylhydrogen siloxane copolymer, and according to the MSDS contains 0.19 wt % xylene, <0.1 wt % ethylbenzene, 55 to 75 wt % dimethyl, methylhydrogen siloxane, 15 to 35 wt % dimethylvinyl-terminated dimethyl siloxane, 10 to 30 wt % dimethylvinylated and trimethylated silica and 1 to 5 wt % tetramethyl tetravinyl cyclotetrasiloxane. The thermal curing is known to be an addition curing system, where component A contains vinyl groups, component B contains Si—H groups and the addition reaction can be catalyzed by a Pt compound (usually hexachloro platinate) which is part of the curing agent. These components are mixed together and then heated to cure the part.

Historically, multilayer PDMS devices have been prohibitively complicated or impossible to assemble due to layer-to-layer registration issues. The process described herein offers layer-to-layer registration repeatability of approximately 1 micrometer.

The research began by trying to find a laser capable of cutting cured PDMS into shape. A laser having a 10.6 micrometer wavelength, as that used in Riahi, was evaluated and it was incapable of cutting the cured PDMS. It appeared as if the glass slide upon which the PDMS part was resting was absorbing the energy and blowing up. A 1.2 micrometer wavelength laser was evaluated. It showed slightly better results but the slide still seemed to heat before the PDMS. A 9.3 micrometer wavelength laser was evaluated and the ability to cut was demonstrated. Seeing the ability to cut the cured PDMS led to the thought that the 9.3 micrometer wavelength might work for thermally curing the PDMS as well.

On the basis of these results it was surprisingly discovered that a much thinner layer could be cured (7 micron) when a 9.3 micrometer wavelength laser was used as opposed to the 200-400 micrometer layer associated with the 10.6 micrometer wavelength used in the prior art.

What was eventually discovered was that the transmission of electromagnetic radiation through PDMS at the wavelength of 9.3 micrometers is much lower than the transmission at a wavelength of 10.6 micrometers. Because transmission is relative to thickness, it is always normalized to a thickness, which this application choses to be 1 micrometer for ease of calculations.

While not to be bound by any theory it is believed that the high transmittance at 10.6 micrometers, as opposed to a low transmission (high absorbance), explains why the cure depth at 10.6 micrometers is as deep as 200-400 micrometers as noted in Riahi. In other words, it is believed that the electromagnetic radiation energy at that wavelength is simply just passing through the PDMS and heating very little of the top layer. The top of the layer(s) of the article are just do not absorb enough of the energy at 10.6 micrometers to thermally cure the material in time.

The incident energy is not limited to a single wavelength source, but could be a range of wavelengths or perhaps two or more discrete wavelengths striking the surface, just so long as the heat curable mixture has a transmission of less than 60% of the focused incident energy (electromagnetic radiation) per micron of the heat curable mixture. It is more preferred that the relative transmission of the focused incident electromagnetic radiation per micrometer of the heat curable mixture be less than 60%, with less than 55% being more preferred, with less than 50% being even more preferred, with less than 45% being even more preferred, with less than 40% also being more preferred, with less than 35% being most preferred.

This can also be stated as being transmittable through 1 micron of the heat curable mixture. In other words, the wavelengths of the focused incident electromagnetic radiation should be selected so that less than 60% of the focused incident electromagnetic radiation per micrometer is transmittable through 1 micron of the heat curable mixture. It is more preferable that less than 55% of the focused incident electromagnetic radiation per micrometer be transmittable through 1 micron of the heat curable mixture. It is even more preferable that less than 50% of the focused incident electromagnetic radiation per micrometer be transmittable through 1 micron of the heat curable mixture, with less than 45% being even more preferred, with less than 40% being even more preferred, with less than 35% being most preferred.

The phrase focused incident electromagnetic radiation is used to distinguish the radiation from room lighting, or other background lighting. Focused incident electromagnetic radiation is directed to a very small area such as a dot having diameter of 80 microns, and is typical of laser or lens apparatus.

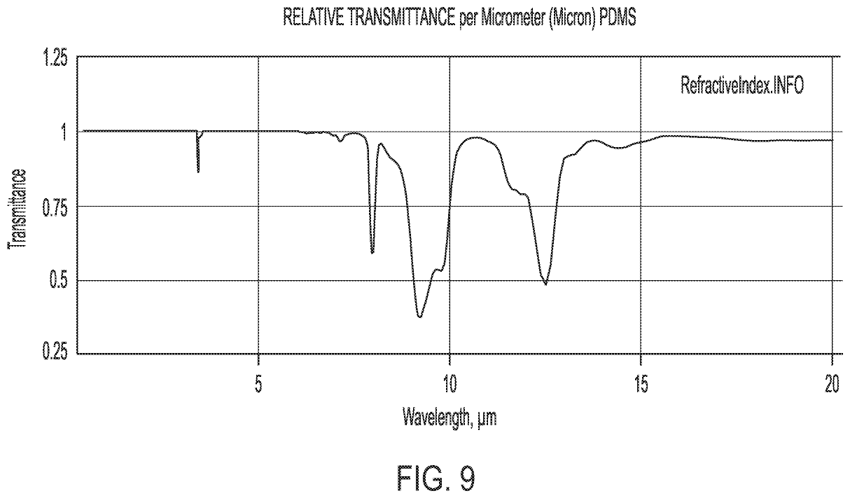

The data of Transmission of electromagnetic radiation vs its wavelength for PDMS show in FIG. 9 can be found at the website https://refractiveindex.info/?shelf=organic&book=polydimethylsiloxane&page=Zhang-10-1. A Transmittance of 0.75 for a 1 micron thick layer is the equivalent of stating that 75% of the incident radiation at that wavelength passes through 1 micron layer. As seen in FIG. 9, the graph is a relative transmission with a value of 1.0 being assigned to the amount of incident radiation (100%). A value of 0.5 means that 50% of the incident radiation striking the surface of the 1 micron thick PDMS will pass through the part.

As shown in FIG. 9 there are 5 Transmission valleys with minima transmissions of 86%@3.4 micrometers, 60%@8 micrometers, 37%@9.3 micrometers, and 48%@12.5 micrometers. As can be seen from FIG. 9 the rest of spectra exhibits more than 75% Transmission, and in many cases almost 100% Transmission.

This relative Transmission per micrometer parameter is useful in both vat printing and spin coat printing as described below.

Once the relationship between transmittance per micrometer and the wavelength of electromagnetic radiation was made, transmittance per micrometer of the heat curable mixture became a control parameter in selecting the wavelengths of the energy source and the associated power. Less transmittance (more absorbance), means a faster cure for thinner parts, thus enabling smaller layers, smaller channels, and better resolution and repeatability of the microfluidic features and structures. For the purposes of this specification it is assumed that the transmission through the heat curable mixture and the thermally cured PDMS part are the same and therefore used interchangeably.

High-angular precision spin coating and laser lithography was used to 3D print PDMS microfluidic devices directly onto microscope slides with layer thickness of ˜10 micrometers (μm) with layers as thin as 5 micrometers achieved. Laser lithography was used to thermally cure the PDMS cross section of each layer to the previous layer.

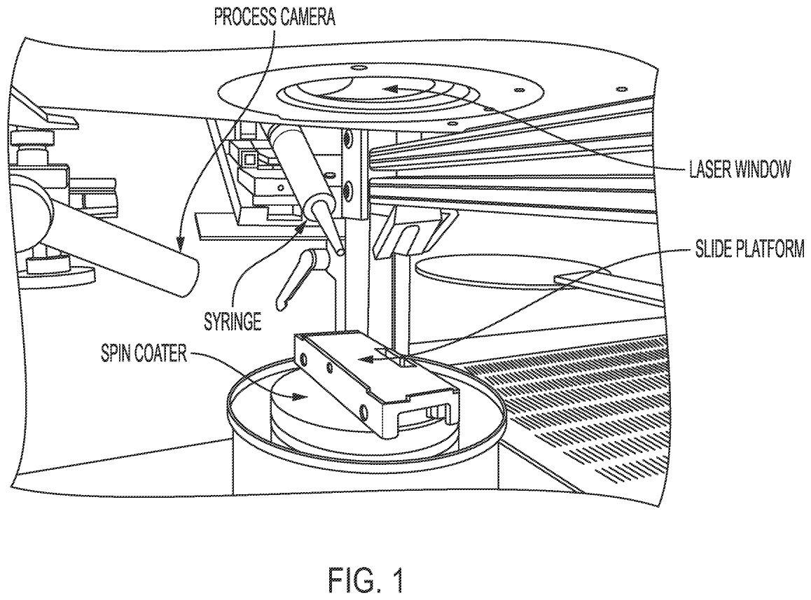

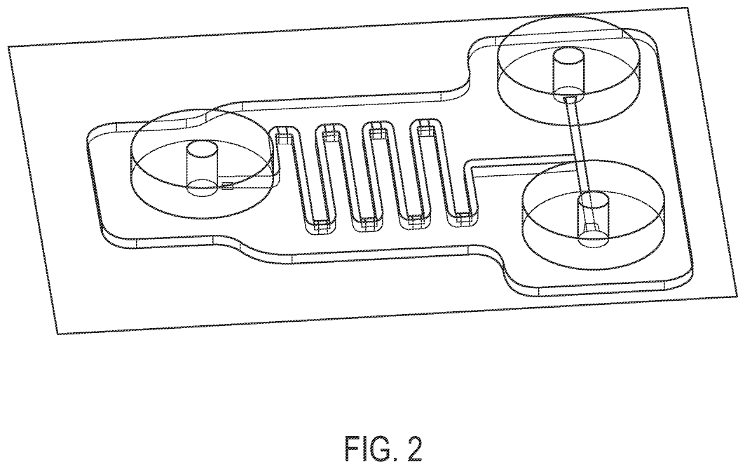

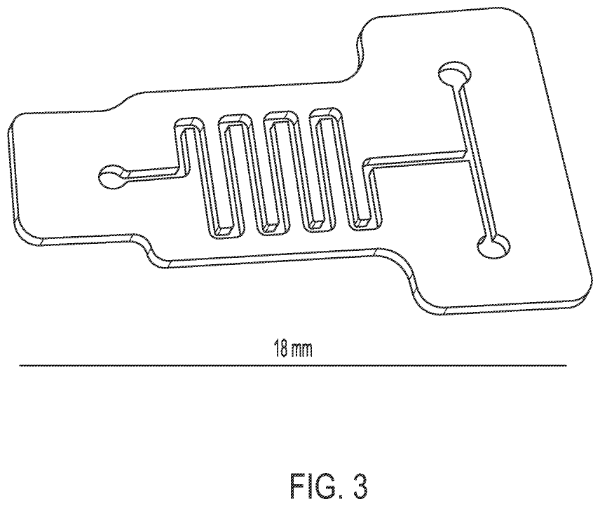

FIG. 1 shows an image of the 3DSC (three dimensional spin coating) unit used to make the static microfluidic mixer shown in FIG. 3 based upon the CAD model of FIG. 2. As can be seen, there is the syringe for placing the heat curable mixture upon the slide or top of the previously cured layer, a laser which emits the focused electromagnetic radiation, and a slide plate that rests on the turntable of the spin coater. The camera allows observation of the process.

The internal channels of this device are ˜250 micrometers wide by ˜60 micrometers tall and are accessed via the three ports in the lid. The layer thickness used to print this device in FIG. 2 and FIG. 3 was 6 micrometers.

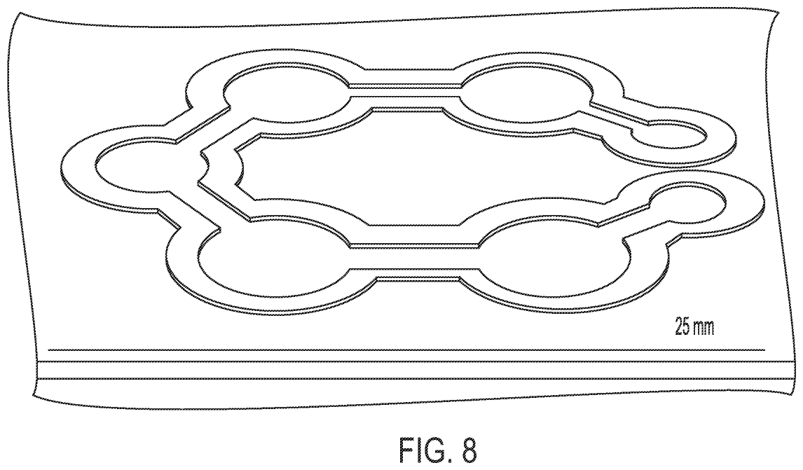

FIG. 8 is a Multi-Organ-On-a-Chip device printed using the disclosed process. It is noted that the bioinks often found in such devices are not present in this example.

Because speed of curing is essential in printing the part, the less the transmission per micrometer, the quicker the part can be built and subsequently cleaned of the uncured PDMS. The manufacturing process must balance rapid curing of the individual layer with the relatively slow curing of the overall mixture. If it takes a long time to cure at room temperature, the part can be built before the internal uncured material, such as that in a channel, can cure and the uncured material can be removed. If the cure at room temperature is too fast, the uncured material will cure before the part is finished. The proper rate was accomplished by using a 10:1 ratio of uncured liquid PDMS to the thermal curing agent (hardener), which could be a mixture of compounds.

PDMS in the standard 10:1 ratio has a relatively long curing time at room temperature which allows the multi-layer PDMS devices to be built over several hours. In the disclosed process spin coating is used to produce the successive layers, and the layers are thermally cured and adhered using a CO₂ laser.

The process begins by depositing and curing a base layer (L0 which is LN where N=0) of PDMS onto a plasma-cleaned microscope slide. This base is approximately 50 micrometers thick.

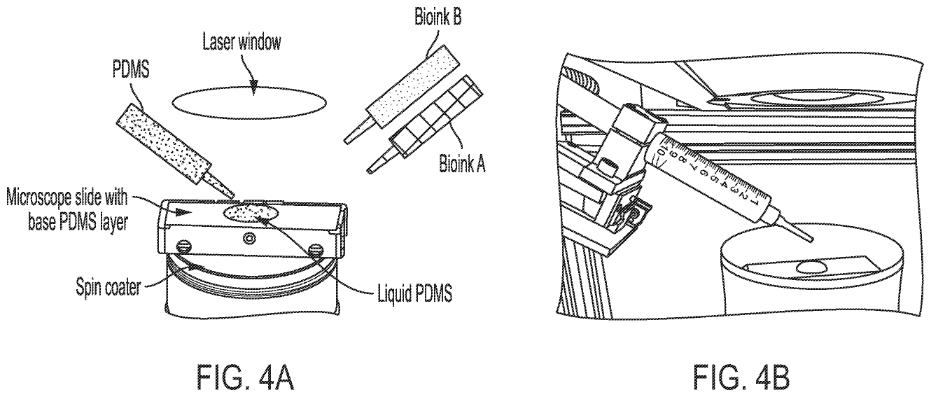

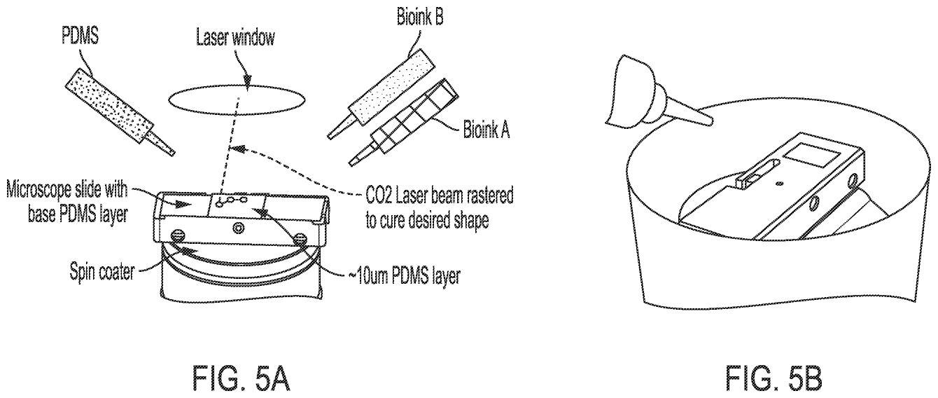

This slide is then loaded onto a slide platform (FIG. 1 and FIG. 4A and FIG. 4B) which clamps only the slide edges. The slide platform sits on a custom-designed and fabricated high precision spin coater which has angular positioning resolution of 5.5×10 degrees and a ball-detent locking mechanism to ensure layer-to-layer registration after spin coating. FIG. 4 and FIG. 5A and FIG. 5B.

To begin the build process, a translating syringe pump (labeled PDMS in the figures) deposits 0.5 ml liquid heat curable mixture of PDMS and the curing agent onto the slide, as shown in FIG. 4. The heat curable mixture on the coated slide is then spun at 4800 RPMs for 60 seconds to form a uniform layer of the heat curable mixture having a thickness such as 15 micrometers or 6 micrometers. The spin-coater angle is then rotated to the patterning position and locked in place. A 9.3 micrometer wavelength CO₂ laser lithography unit then emits a focused beam to selectively cure portions of the heat curable mixture into the desired layer geometry by moving the beam in a predefined pattern that corresponds to the desired cross-section as shown FIG. 5A and FIG. 5B.

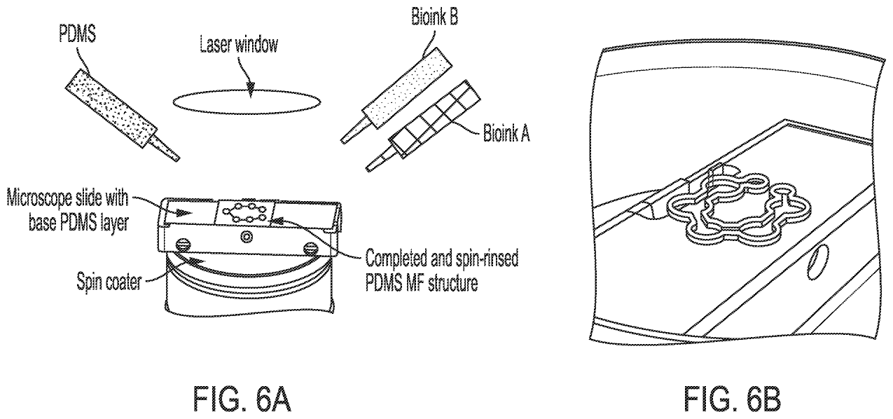

Upon completion of the laser curing, the syringe pump deposits another drop of the heat curable mixture containing PDMS on the top layer; the newly deposited drop is spun into another 6 micrometer thick layer which is then laser cured. Subsequent layers of cured PDMS are made by depositing a drop of the heat curable mixture onto the top of the device, spinning the drop into a layer, and then curing with a laser until the cured PDMS structure of the MOOC (Multi Organ On a Chip) is completed as shown in FIG. 6A. The image on the right (FIG. 6B) shows such a 3DSC printed PDMS structure.

The device or article is built when all the layers are selectively cured and there are no more layers to be placed on the device. Once built, the uncured PDMS is removed using techniques known in the art. In this case, the uncured PDMS remaining around the cured microfluidic will be removed by flooding the spin coater with isopropanol and spin cleaning away the liquid and dissolved uncured heat curable mixture containing PDMS. The internally contained uncured heat curable mixture is removed by flushing isopropanol or other suitable solvent through the various features, such as a channel. Additionally, the PDMS microfluidic is post cured as needed.

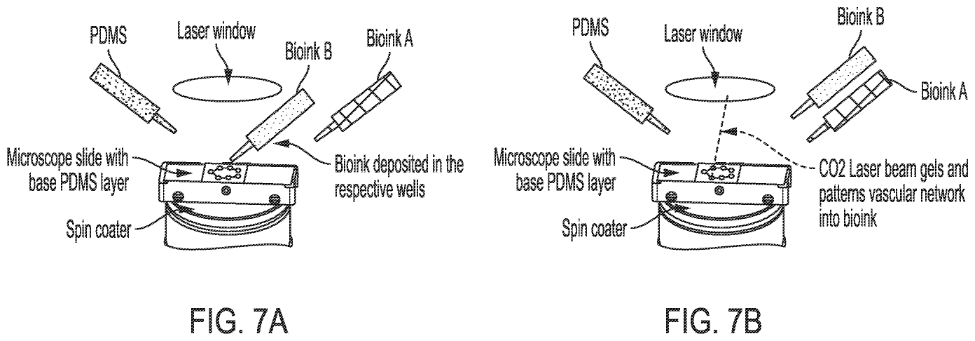

After cleaning and post-curing the PDMS microfluidic, bioinks can be deposited into the respective wells and then gelled and patterned with the laser to form a vascular network in each of the bioinks as shown in FIGS. 7A and 7B. Upon completion of the gelation and patterning, the device will be incubated, and the polymer used for gelation removal and further perfused as needed.

The second approach to using 3DSC to process bioink structures is to print freestanding multilayer structures by successively spin coating, gelling, and patterning bioinks in a prescribed order. This method will allow for the formation of layered network structures to study complex multilayer biological networks including vascular structures without the presence of external confinement.

In this process the current layer being spread and cured is the current layer (LN). The first layer in contact with the slide is the base layer L(0). The layer immediately previous to the current layer is L(N−1). Each layer will have a layer thickness, which is denoted T(N).

As mentioned previously, the PDMS of this invention can be free of UV adsorber(s), photo-initiators, or other agents overcoming the inherent difficulties of current curing techniques. Put another way, the heat curable mixture can be considered to have an amount of PDMS and an amount of heat curing agent (hardener), which on a weight basis add up to 100 weight percent of the heat curable mixture. The heat curing agent could be a mixture of components blended with the liquid uncured PDMS. However, the heat curable mixture should be void of any substance that does not play a role in the thermal curing or thermal hardening of the PDMS, such as a photo-initiator like a UV absorber.

Because the mixture is void of a UV photo-initiator, The mixture and cured part can be described as having a transmission of greater than 90% per micron in the UVA (320-395 nm) and UVB (280-320 nm) spectra, with greater than 95% per micron more preferred.

It was later discovered that by using a CO₂ laser at 9.3 micrometer, layers as small as 1 micrometer and less could be cured. This enables the microfluidic PDMS part to have channels with depths less than 200 micrometer, more particularly in the range of 50 to 200 micrometer in depth, 25 to 150 microns being the most preferred depth.

The final part made from this method may comprises a channel having at least one dimension relative to the base layer selected from the group consisting of a height and a width that is less than a value selected from the group consisting of 200 micrometers, 150 micrometers, 100 micrometers, 75 micrometers, and 50 micrometers.

Typical ranges for layer thickness of this invention are 5 micrometers to 200 micrometers, 5 micrometers to 150 micrometers, 5 micrometers to 100 micrometers, and 5 micrometers to 50 micrometers. Alternatively, this can be recited as the article comprises at least one layer having a thickness selected from the group consisting of 5 micrometers to 200 micrometers, 5 micrometers to 150 micrometers, 5 micrometers to 100 micrometers, and 5 micrometers to 50 micrometers.

Other typical ranges of layer thickness of this invention are 1 micrometers to 200 micrometers, 1 micrometers to 150 micrometers, 1 micrometers to 100 micrometers, and 1 micrometers to 50 micrometers. Alternatively, this can be recited as the article comprises at least one layer having a thickness selected from the group consisting of 1 micrometers to 200 micrometers, 1 micrometers to 150 micrometers, 1 micrometers to 100 micrometers, and 1 micrometers to 50 micrometers.

1-17. (canceled)

18. A method of additively manufacturing a multilayered heat cured article from a thermally curable mixture comprising a polymer and a thermal curing agent;
the method comprising;
creating a cured base layer with a thickness of at least 50 micrometers of the polymer comprising the thermally curable mixture;
forming a heat curable current layer (LN) comprising the thermally curable mixture having a heat curable current layer thickness (TN) on a preceding layer (L(N−1));
curing at least a portion of the heat curable current layer by selectively applying an incident electromagnetic radiation energy to heat at least a portion of the heat curable current layer and optionally leaving an uncured portion of the heat curable current layer, wherein less than 50% of the incident electromagnetic radiation energy is transmittable through 1 micron of the thermally curable mixture;
repeating steps B to C until the multilayered heat cured article is built; and
removing the uncured portions from inside and around the multilayered heat cured article.

19. The method of claim 18, wherein the incident electromagnetic radiation energy has a wavelength in a range of 9.2 to 9.4 micrometers.

20. The method of claim 18, wherein the polymer is polydimethylsiloxane (PDMS).

21. The method of claim 20, wherein the incident electromagnetic radiation energy has a wavelength in a range of 9.2 to 9.4 micrometers.

22. The method of claim 20, wherein the thermal curing agent contains methylhydrosiloxane and a platinum catalyst.

23. The method of claim 22, wherein the incident electromagnetic radiation energy has a wavelength in a range of 9.2 to 9.4 micrometers.

24. An article of manufacture comprised of a thermally cured polymer, wherein the article is multi-layered, having a layer thickness in a range of 1 micrometer to 100 micrometers.

25. The article of claim 24, wherein the thermally cured polymer is thermally cured PDMS.

26. The article of claim 24, wherein the article is void of a photo-initiator, photo catalysts and other additives used for UV curing of polymers.

27. The article of claim 24, wherein the thermally cured polymer comprising the article has been thermally cured using electromagnetic radiation in a wavelength range of 9.2 to 9.4 micrometers.

28. The article of claim 25, wherein the article is void of a photo-initiator, photo catalysts and other additives used for UV curing of polymers.

29. The article of claim 25, wherein the thermally cured polymer comprising the article has been thermally cured using electromagnetic radiation in a wavelength range of 9.2 to 9.4 micrometers.

30. The article of claim 26, wherein the thermally cured polymer comprising the article has been thermally cured using electromagnetic radiation in a wavelength range of 9.2 to 9.4 micrometers.
